(19) United States
(12) Patent Application Publication
(10) Pub. No.: US 2026/0137730 A1
(43) Pub. Date: May 21, 2026

(54) COMPOSITION CONTAINING EXTRACT OF AKKERMANSIA MUCINIPHILA BAA-835 AND METHOD THEREOF

(71) Applicant: Leeuwenhoek Laboratories Co. Ltd., Taipei City (TW)

(72) Inventors: Chiao-Wei LIN, Taipei City (TW); Yi-Ling LO, Taipei City (TW); Hsin-Bai ZOU, Taipei City (TW); Wen-Hui LIN, Taipei City (TW); Chi-Shan LI, Taipei City (TW); Cheng-Chih HSU, Taipei City (TW)

(21) Appl. No.: 19/392,170

(22) Filed: Nov. 18, 2025

Related U.S. Application Data
(60) Provisional application No. 63/721,573, filed on Nov. 18, 2024.

(51) Int. Cl. A61K 35/74; A61P 3/04; C12N 1/20

(52) U.S. Cl. CPC A61K 35/74; A61P 3/04; C12N 1/20

(57) ABSTRACT

An extract of Akkermansia muciniphila BAA-835 is provided, comprising: providing a bacterial culture medium containing Akkermansia muciniphila BAA-835; centrifuging the bacterial culture medium to obtain a supernatant and a precipitate; extracting a portion of the supernatant with ethyl acetate to obtain an ethyl acetate aqueous layer; drying the ethyl acetate aqueous layer to obtain an aqueous ethyl acetate extract; extracting a remaining portion of the supernatant with n-hexane to obtain a n-hexane aqueous layer and a n-hexane organic layer; and drying the n-hexane aqueous layer and the n-hexane organic layer respectively to obtain the aqueous n-hexane extract and the organic n-hexane extract. Also provided is a method of the extract as described above for treating and preventing overweight or obesity disease, increasing GLP-1, lowering triglycerides, or combinations thereof.

### CROSS-REFERENCE TO RELATED APPLICATION

This application claims priority to U.S. Provisional Application Ser. No. 63/721,573, filed on Nov. 18, 2024, which is herein incorporated by reference in its entirety.

### BACKGROUND

### Field of Invention

The present invention relates to a composition containing extract of Akkermansia muciniphila BAA-835 and method thereof.

### Description of Related Art

Since 1975, the global prevalence of obesity has almost tripled, primarily because of unhealthy eating habits. Since obesity disease is associated with increased susceptibility to various chronic diseases, the growing prevalence of overweight has become a significant global public health issue. While a considerable number of weight-loss drugs are available, not all of them are effective for obese subjects. Because of the numerous causes of obesity, some obese subjects may have glucagon-like peptide-1 (GLP-1) deficiency.

Therefore, the related art really needs to be improved.

### SUMMARY

One embodiment of the present disclosure provides a composition, comprising: an extract of Akkermansia muciniphila BAA-835 prepared by the following steps comprising: providing a bacterial culture medium, wherein the bacterial culture medium comprising Akkermansia muciniphila BAA-835 (ATCC BAA-835); centrifuging the bacterial culture medium to obtain a supernatant and a precipitate; extracting a portion of the supernatant with ethyl acetate to obtain an ethyl acetate aqueous layer; drying the ethyl acetate aqueous layer to obtain an ethyl acetate aqueous layer extract; extracting a remaining portion of the supernatant with n-hexane to obtain a n-hexane aqueous layer and a n-hexane organic layer; and drying the n-hexane aqueous layer and the n-hexane organic layer respectively to obtain a n-hexane aqueous layer extract and a n-hexane organic layer extract; and a pharmaceutical or food-acceptable carrier.

In some embodiments, the composition is a food composition or a pharmaceutical composition.

In some embodiments, the composition is formulated into capsules, tablets, powders or liquids.

Another one embodiment of the present disclosure provides a method of preventing and treating overweight or obesity disease comprising administering to a subject in need thereof an effective amount of a composition, wherein the composition comprises an extract of Akkermansia muciniphila BAA-835, wherein the extract of Akkermansia muciniphila BAA-835 prepared by the following steps comprises: providing a bacterial culture medium, wherein the bacterial culture medium comprises Akkermansia muciniphila BAA-835 (ATCC BAA-835); centrifuging the bacterial culture medium to obtain a supernatant and a precipitate; extracting a portion of the supernatant with ethyl acetate (EA) to obtain an ethyl acetate aqueous layer; drying the ethyl acetate aqueous layer to obtain an ethyl acetate aqueous layer extract; extracting a remaining portion of the supernatant with n-hexane (Hex) to obtain a n-hexane aqueous layer and a n-hexane organic layer; and drying the n-hexane aqueous layer and the n-hexane organic layer respectively to obtain a n-hexane aqueous layer extract and a n-hexane organic layer extract.

In some embodiments, the composition is a food composition or a pharmaceutical composition.

In some embodiments, the composition is formulated into capsules, tablets, powders or liquids.

In some embodiments, the composition is administered to a subject orally or parenterally.

In some embodiments, the overweight or the obesity disease is caused by diet.

In some embodiments, the method of preventing and treating overweight or obesity disease further comprising increasing GLP-1 level.

In some embodiments, the method of preventing and treating overweight or obesity disease further comprising reducing triglyceride level.

In some embodiments, the composition further comprises a pharmaceutical or food-acceptable carrier.

Another one embodiment of the present disclosure provides a method for increasing GLP-1, reducing triglyceride, or a combination thereof, comprising administering to a subject in need thereof an effective amount of a composition, wherein the composition comprising an extract of Akkermansia muciniphila BAA-835, wherein the extract of Akkermansia muciniphila BAA-835 prepared by the following steps comprises: providing a bacterial culture medium, wherein the bacterial culture medium comprises Akkermansia muciniphila BAA-835 (ATCC BAA-835); centrifuging the bacterial culture medium to obtain a supernatant and a precipitate; extracting a portion of the supernatant with ethyl acetate (EA) to obtain an ethyl acetate aqueous layer; drying the ethyl acetate aqueous layer to obtain an ethyl acetate aqueous layer extract; extracting a remaining portion of the supernatant with n-hexane (Hex) to obtain a n-hexane aqueous layer and a n-hexane organic layer; and drying the n-hexane aqueous layer and the n-hexane organic layer respectively to obtain a n-hexane aqueous layer extract and a n-hexane organic layer extract.

In some embodiments, the composition is a food composition or a pharmaceutical composition.

In some embodiments, the composition is formulated into capsules, tablets, powders or liquids.

In some embodiments, the composition is administered to a subject orally or parenterally.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 depicts testing the differences in blood GLP-1 secretion in different groups of mice after glucose ingestion according to some embodiments of the present disclosure. T-test, *refers to p<0.05, **refers to p<0.01.

FIG. 2 depicts the difference in triglyceride (TG) concentration in mice from different groups of mice according to some embodiments of the present disclosure. T-test, *refers to p<0.05, **refers to p<0.01.

FIG. 3 depicts the differences in GLP-1 secretion after different extract treatments of NCI-H716 cells according to some embodiments of the present disclosure. T-test, *refers to p<0.05, **refers to p<0.01. The leftmost column shows the negative control group, NCI-H716 cells, added only with an equal volume of dimethyl sulfoxide (DMSO) and HBSS containing 0.2% BSA. Con: extract of uncultured medium. CA7S: 1 μM cholic acid-7-sulfate as the positive control group. Akk: extract of Akkermansia muciniphila BAA-835bacterial culture.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number. This repetition is for the purpose of simplicity and clarity and does not in itself dictate a relationship between the various embodiments and/or configurations discussed.

As used herein, obesity refers to a physical state in which excessive accumulation of body fat has a negative impact on health. The main definition of obesity is based on the body mass index (BMI (kg/m²)). The obesity standards were established by the World Health Organization in 1997 and published in 2000. The detailed standards are as follows: overweight BMI is 25.0˜29.9, obesity (grade I) BMI is 30.0˜34.9, obesity (grade II) BMI is 35.0˜39.9, and obesity (grade III) BMI is ≥40.0.

As used herein, “overweight” refers to that BMI is from 25.0 to 29.9.

As used herein, “obesity disease” refers to that a BMI is 30.0 or above, for example, obesity (grade I), obesity (grade II), or obesity (grade III).

Since there are many causes of obesity, some obese subjects may be patients with GLP-1 deficiency. Supplementing with GLP-1 can slow down the peristalsis of the intestines and reduce the digestion rate, thereby reducing the rate of energy intake and achieving the ideal effect of weight loss.

As used herein, “Akkermansia muciniphila BAA-835” is a known freely distributable strain, deposited at the American Type Culture Collection (ATCC) with the catalog number ATCC BAA-835. The standard strain ATCC BAA-835 was described by Muriel Derrien et al. in 2004 as Gram-negative, anaerobic, and approximately 0.6 μm to 1.0 μm in size.

Some embodiments of the present disclosure provide an extract of Akkermansia muciniphila BAA-835 prepared by the following steps comprising: providing a bacterial culture medium, wherein the bacterial culture medium comprising Akkermansia muciniphila BAA-835 (ATCC BAA-835); centrifuging the bacterial culture medium to obtain a supernatant and a precipitate; extracting a portion of the supernatant with ethyl acetate (EA) to obtain an ethyl acetate aqueous layer; drying the ethyl acetate aqueous layer to obtain an ethyl acetate aqueous layer extract; extracting a remaining portion of the supernatant with n-hexane (Hex) to obtain a n-hexane aqueous layer and a n-hexane organic layer; and drying the n-hexane aqueous layer and the n-hexane organic layer respectively to obtain a n-hexane aqueous layer extract and a n-hexane organic layer extract.

Another embodiment of the present disclosure provides a composition containing an extract of Akkermansia muciniphila BAA-835, comprising: the extract of Akkermansia muciniphila BAA-835 as above mentioned, comprising the ethyl acetate aqueous layer extract, the n-hexane aqueous layer extract, or the n-hexane organic layer extract; and a pharmaceutical or food-acceptable carrier.

In some embodiments, the composition is administered to a subject orally or parenterally. In some embodiments, the composition is formulated into an oral dosage form selected from the group consisting of solution, suspension, emulsion, powder, lozenge, pill, syrup, buccal lozenge, tablet, chewing gum and capsule for administration to a subject.

In some embodiments, the pharmaceutical or food-acceptable carrier includes, but is not limited to, water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, and other similar or applicable carriers for the present invention.

In some embodiments, the composition may be a food composition. For example, added to edible materials in the form of food additives to prepare a food product for human or animal consumption. Food composition includes, but is not limited to, general foods, health foods, beverages, nutritional supplements, dairy products or feeds, etc. In some examples of oral dosage forms, the composition may optionally include pharmaceutical and food-acceptable carriers, excipients and/or additives. In other examples, the dosage form of the synbiotic composition may include, but is not limited to, powders, tablets, granules, suppositories, microcapsules, ampoules, liquid sprays or suppositories.

In some embodiments, the composition applies to obese individuals. In some examples, obese individuals are selected from the group consisting of caused by diet, type 2 diabetes, hyperglycemia, glucose intolerance, dyslipidemia, insulin resistance, hyperinsulinemia, fatty liver, cardiovascular disease, stroke, cancer, and a combination thereof.

Another embodiment of the present disclosure provides a use of the extract of Akkermansia muciniphila BAA-835 as above mentioned for the manufacture of a composition for preventing and treating overweight or obesity disease, wherein the extract of Akkermansia muciniphila BAA-835 comprises the ethyl acetate aqueous layer extract, the n-hexane aqueous layer extract, or the n-hexane organic layer extract.

Another embodiment of the present disclosure provides a composition for use in preventing and treating overweight or obesity disease, the composition comprises the extract of Akkermansia muciniphila BAA-835 as above mentioned, the extract comprises the ethyl acetate aqueous layer extract, the n-hexane aqueous layer extract, or the n-hexane organic layer extract.

In some examples, the composition further comprises a pharmaceutical or food-acceptable carrier.

Another embodiment of the present disclosure provides a use of the extract of Akkermansia muciniphila BAA-835 as above mentioned for the manufacture of a composition for increasing GLP-1, wherein the extract of Akkermansia muciniphila BAA-835 comprises the ethyl acetate aqueous layer extract, the n-hexane aqueous layer extract, or the n-hexane organic layer extract.

Another embodiment of the present disclosure provides a composition for use in increasing GLP-1, the composition comprises the extract of Akkermansia muciniphila BAA-835 as above mentioned, the extract comprises the ethyl acetate aqueous layer extract, the n-hexane aqueous layer extract, or the n-hexane organic layer extract.

In some examples, the composition further comprises a pharmaceutical or food-acceptable carrier.

In some embodiments, a preparation method of the extract of Akkermansia muciniphila BAA-835 is that Akkermansia muciniphila BAA-835 strain was cultured in suspension with a basal mucin-based medium until the OD₆₀₀=0.5˜0.6. Then, 75 mL of the bacterial suspension was inoculated into 3 L of the basal mucin-based medium and cultured for 14 hours to obtain a bacterial culture medium. In some examples, when cultured to OD₆₀₀=0.5˜0.6, the number of Akkermansia muciniphila BAA-835 strains was approximately 2×10⁸ CFU/mL to 8×10⁸ CFU/mL, for example, 2×10⁸ CFU/mL, 3×10⁸ CFU/mL, 4×10⁸ CFU/mL, 5×10⁸ CFU/mL, 6×10⁸ CFU/mL, 7×10⁸ CFU/mL, 8×10⁸ CFU/mL, or any value between any two of these values. Next, the bacterial culture medium was centrifuged to obtain a precipitate containing bacteria and a supernatant. In some examples, the purpose of centrifugation is to initially isolate and remove the bacterial strain while retaining the secretions and metabolites produced during the bacterial culture process; therefore, any conventional centrifugation speed and time that can achieve this effect is also included. Centrifugation conditions include, but are not limited to, 10,000×g (RCF) for 30 minutes. Next, the supernatant can be selectively filtered through a 0.22 μm filter membrane to remove any remaining bacterial strains. In some examples, the centrifuged supernatant may contain no or a small amount of bacterial strains; if a small amount is present, further filtration can be used to remove the remaining strains. Then, a portion of the supernatant was extracted with ethyl acetate (EA) to obtain an ethyl acetate aqueous layer (EA/H₂O) and an ethyl acetate organic layer (EA/EA). In some examples, a volume percentage of ethyl acetate includes, but is not limited to, 5%, 10%, 20%, 30%, 40%, 50%, 60%, 70%, 80%, 90%, 95%, 99.5%, or any value between any two of these values. The types of substances extracted from the ethyl acetate aqueous layer with different volume percentages of ethyl acetate were roughly the same, and the types of substances extracted from the ethyl acetate organic layer with different volume percentages of ethyl acetate were roughly the same. The only difference was that a higher volume percentage of ethyl acetate resulted in a larger volume of ethyl acetate organic layer, and the more amount of non-polar metabolites can be extracted (with the same types of substances), while a smaller volume of ethyl acetate aqueous layer resulted in less amount of polar metabolites can be extracted (with the same types of substances), and vice versa. The remaining portion of the supernatant was extracted with n-hexane (Hex) to obtain a n-hexane aqueous layer (Hex/H₂O) and a n-hexane organic layer (Hex/Hex). In some examples, a volume percentage of n-hexane includes, but is not limited to, 5%, 10%, 20%, 30%, 40%, 50%, 60%, 70%, 80%, 90%, 95%, 99.5%, or any value between any two of these values. The types of substances extracted from n-hexane aqueous layers with different volume percentages of n-hexane were roughly the same, and the types of substances extracted from the n-hexane organic layers with different volume percentages of n-hexane were roughly the same. The only difference is that a higher volume percentage of n-hexane resulted in a larger volume of n-hexane organic layer, and the more amount of non-polar metabolites can be extracted (with the same types of substances), while a smaller volume of n-hexane aqueous layer resulted in less amount of polar metabolites can be extracted (with the same types of substances), and vice versa.

One embodiment of the present disclosure also provide a use (application) of an extract of Akkermansia muciniphila BAA-835 for the manufacture of a composition for preventing or treating reduced GLP-1 secretion.

One embodiment of the present disclosure also provides a use (application) of an extract of Akkermansia muciniphila BAA-835 for the manufacture of a composition for preventing or treating GLP-1 insufficiency.

One embodiment of the present disclosure also provides a use (application) of an extract of Akkermansia muciniphila BAA-835 for the manufacture of a composition for preventing or treating GLP-1 deficiency.

One embodiment of the present disclosure also provide a use of the extract of Akkermansia muciniphila BAA-835 as above mentioned for the manufacture of a composition for reducing triglyceride, in which the extract of Akkermansia muciniphila BAA-835 comprises the ethyl acetate aqueous layer extract, the n-hexane aqueous layer extract, or the n-hexane organic layer extract.

One embodiment of the present disclosure also provide a composition for use in reducing triglyceride, comprising the extract of Akkermansia muciniphila BAA-835 as above mentioned, the extract comprises the ethyl acetate aqueous layer extract, the n-hexane aqueous layer extract, or the n-hexane organic layer extract.

In some examples, the composition further comprises a pharmaceutical or food-acceptable carrier.

A number of examples are provided herein to elaborate the composition of the instant disclosure. However, the examples are for demonstration purpose alone, and the instant disclosure is not limited thereto.

A number of examples are provided herein to elaborate the extract of Akkermansia muciniphila BAA-835 of the instant disclosure. However, the examples are for demonstration purpose alone, and the instant disclosure is not limited thereto.

For the sake of clarity, features and elements that are well known in the art and are not necessary for an understanding of the principles described have been omitted.

### Preparation Example 1

Method of preparing extract of Akkermansia muciniphila BAA-835.

Akkermansia muciniphila BAA-835 strain was cultured in suspension with a basal mucin-based medium (Plovier et al., Nature Medicine, 2017) until the OD₆₀₀=0.5˜0.6. Then, 75 mL of the bacterial suspension was inoculated into 3 L of the basal mucin-based medium and cultured for 14 hours to obtain a bacterial culture medium. Next, the bacterial culture medium was centrifuged at 10,000×g (RCF) for 30 minutes to obtain a precipitate containing bacteria and a supernatant and a precipitate, in which the precipitate contains the strain and is removed and only the supernatant is collected. Next, a portion (a half) of the supernatant was extracted with 99.5% ethyl acetate (EA) to obtain an ethyl acetate aqueous layer (EA/H₂O) and an ethyl acetate organic layer (EA/EA). Next, the ethyl acetate aqueous layer (EA/H₂O) and the ethyl acetate organic layer (EA/EA) were dried separately to obtain an ethyl acetate aqueous layer extract and an ethyl acetate organic layer extract, respectively.

The remaining portion (the other half) of the supernatant was extracted with 95% n-hexane (Hex) to obtain a n-hexane aqueous layer (Hex/H₂O) and a n-hexane organic layer (Hex/Hex). Next, the n-hexane aqueous layer (Hex/H₂O) and the n-hexane organic layer (Hex/Hex) were dried separately to obtain a n-hexane aqueous layer extract and a n-hexane organic layer extract.

The control group, which culture medium had not been cultured with any bacteria, was also extracted and dried in the same way as described above. The Hex/Hex extract in the control group was extremely low in content after drying, so it could only be used for the cell experiments in Example 3 at the same dosage in the experimental group, and was not sufficient for the mouse experiments in Example 1 and Example 2.

600 mg of extract from each group was dissolved in 0.1 mL of dimethyl sulfoxide (DMSO), and then diluted 1,000 times with phosphate-buffered saline (PBS) for use in mouse experiments in Examples 1 and 2. 4 mg of extract from each group was dissolved in 0.1 mL of dimethyl sulfoxide (DMSO) for use in cell experiments in Example 3.

### Example 1

Eight-week-old male C57BL/6 rats were used in this experiment and randomly assigned to seven groups: control group (Con), high-fat group (HF), high-fat group+Hex/H₂O-con (n-hexane aqueous layer extract from a culture medium that had not been cultured with any bacteria), high-fat group+Hex/H₂O-AKK (n-hexane aqueous layer extract from a culture medium that had been cultured with Akkermansia muciniphila BAA-835), high-fat group+EA/H₂O-con (ethyl acetate aqueous layer extract from a culture medium that had not been cultured with any bacteria), high-fat group+EA/H₂O-AKK (ethyl acetate aqueous layer extract from a culture medium that had been cultured with Akkermansia muciniphila BAA-835), and high-fat group+Hex/Hex-AKK (n-hexane organic layer extract from a culture medium that had been cultured with Akkermansia muciniphila BAA-835). The control group received a daily control diet (D12450Ji, Research Diet, Inc.), while the high-fat group received a daily high-fat diet (D12492i, 60% fat, Research Diet, Inc.). The high-fat group received Hex/H₂O-con (40 mg extract/kg body weight), Hex/H2O-AKK (40 mg extract/kg body weight), EA/H₂O-con (40 mg extract/g body weight), EA/H₂O-AKK (40 mg extract/g body weight), and Hex/Hex-AKK (25 mg extract/g body weight because the amount of Hex/Hex extract was not enough). In addition to the daily high-fat diet, different extracts were administered daily by oral gavage. The extracts were dissolved in DMSO and diluted with PBS (as shown in the Preparation Example 1 as described), while the control group (Con) and the high-fat group (HF) were given an equal volume of PBS containing 0.1% DMSO daily.

At week 8 of the experiment, blood samples were collected and the concentrations of GLP-1 and triglyceride (TG) in the plasma were analyzed. To compare the differences in GLP-1 concentration in mice from different groups, mice were given glucose solution, and the concentration of GLP-1 in the blood was detected after glucose ingestion. The experimental procedure was as follows: at week 8, mice were fasted for 5 hours and then administered glucose solution by oral gavage at a dose of 2 g/kg BW. Blood was collected 10 minutes later, and a DPP4 inhibitor was added to prevent GLP-1 degradation. After separating plasma from the blood samples, the GLP-1 content in the samples was analyzed using an ELISA kit.

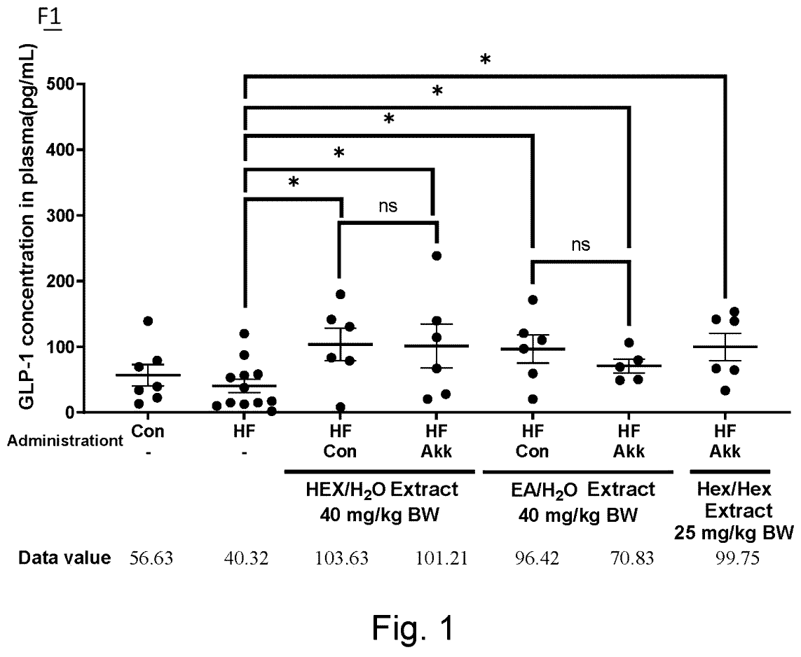

The results are shown in drawing F1 of FIG. 1, the differences in blood GLP-1 secretion in mice of different groups were tested after glucose ingestion. The blood GLP-1 concentration in the control group was 56.63 pg/mL, the blood GLP-1 concentration in the high-fat group was 40.32 pg/mL, the blood GLP-1 concentration in the high-fat group+Hex/H₂O-con was 103.63 pg/mL, the blood GLP-1 concentration in the high-fat group+Hex/H₂O-AKK was 101.21 pg/mL, the blood GLP-1 concentration in the high-fat group+EA/H₂O-con was 96.42 pg/mL, the blood GLP-1 concentration in the high-fat group+EA/H₂O-AKK was 70.83 pg/mL, and the blood GLP-1 concentration in the high-fat group+Hex/Hex-AKK was 99.75 pg/mL. Statistical analysis showed that the blood GLP-1 concentration in the high-fat group+Hex/Hex-AKK was significantly higher than that in the high-fat group. Furthermore, since the Hex/Hex extract in the control group after drying is extremely low yield, which was insufficient to reach the dose of 40 mg/kg BW. Thus, the effect of promoting GLP-1 secretion was mainly derived from Hex/Hex-AKK rather than the culture medium itself.

### Example 2

The experimental groups and procedures were the same as in Example 1. To compare the differences in triglyceride (TG) concentration in mice of different groups, mice were fasted for 6 hours in week 8, blood was collected and plasma was separated, and the TG content in the plasma was analyzed using a TG analysis kit.

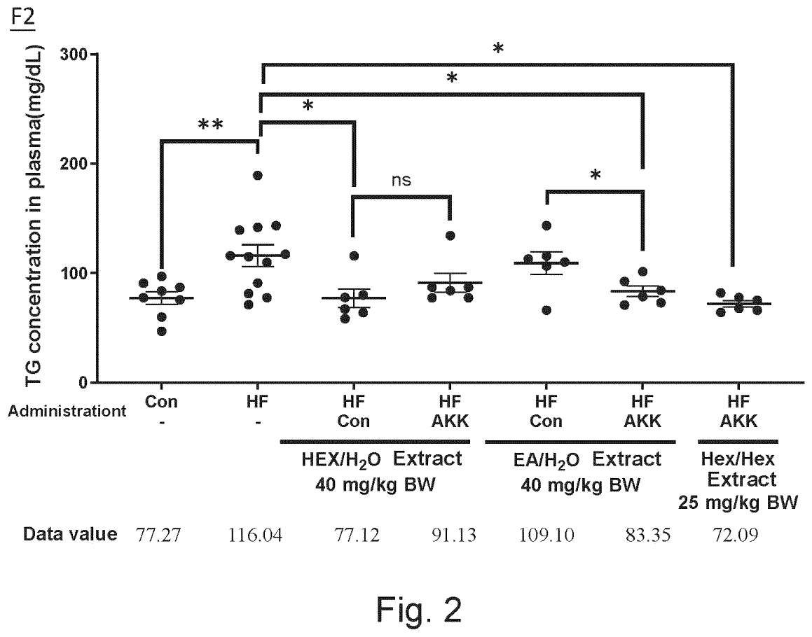

The results are shown in drawing F2 of FIG. 2, the plasma TG concentration in the control group was 77.27 pg/mL, the plasma TG concentration in the high-fat group was 116.04 pg/mL, the plasma TG concentration in the high-fat group+Hex/H₂O-con was 77.12 pg/mL, the plasma TG concentration in the high-fat group+Hex/H₂O-AKK was 91.13 pg/mL, the plasma TG concentration in the high-fat group+EA/H₂O-con was 109.10 pg/mL, the plasma TG concentration in the high-fat group +EA/H2O-AKK was 83.35 pg/mL, and the plasma TG concentration in the high-fat group+Hex/Hex-AKK was 72.09 pg/mL. Statistical analysis showed that the plasma TG concentration in the high-fat group+Hex/Hex-AKK was significantly lower than that of the high-fat group. This indicates that Hex/Hex-AKK has the effect of inhibiting the concentration of TG in the blood.

### Example 3

The NCI-H716 cell line was used as a cell model (this cell line was extracted from colorectal adenocarcinoma ascites and is currently used as a human model for in vitro research on GLP-1 regulation). The NCI-H716 cell line was cultured in RPMI 1640 medium containing 10% fetal bovine serum. Next, the cells were then seeded on a Matrigel®-coated 96-well plate at a density of 6 ×10⁴ cells/well. After culturing the cells on the Matrigel®-coated 96-well plate for 2 days, experiments were performed. Next, the cell culture medium was then removed, and the cells were washed once with Hank's balanced salt solution (HBSS). Next, the HBSS was then removed, and HBSS containing 200 μL of 0.2% BSA was added to the cells. After 2 hours of treatment, the 0.2% BSA HBSS was removed. Next, HBSS containing 0.2% BSA of different extracts from Preparation Example 1 was added to the experimental groups (Hex/Hex extract was 20 μg/mL, each of the three (Hex/H₂O, EA/EA, and EA/H₂O) extracts had two concentrations (20 μg/mL and 200 μg/mL)). The control group (Con) was treated with different extracts from uncultured medium. The negative control group (−) was treated with the same volume of DMSO and HBSS containing 0.2% BSA for 2 hours. Next, the cell culture medium was collected, and Diprotin A (final concentration 10 μM) was added (Note: Diprotin A is a DPP4 inhibitor, inhibiting the DPP4 enzyme to prevent GLP-1 from being degraded by DPP4). Finally, the GLP-1 concentration in the cell culture medium of different groups was analyzed using an ELISA kit.

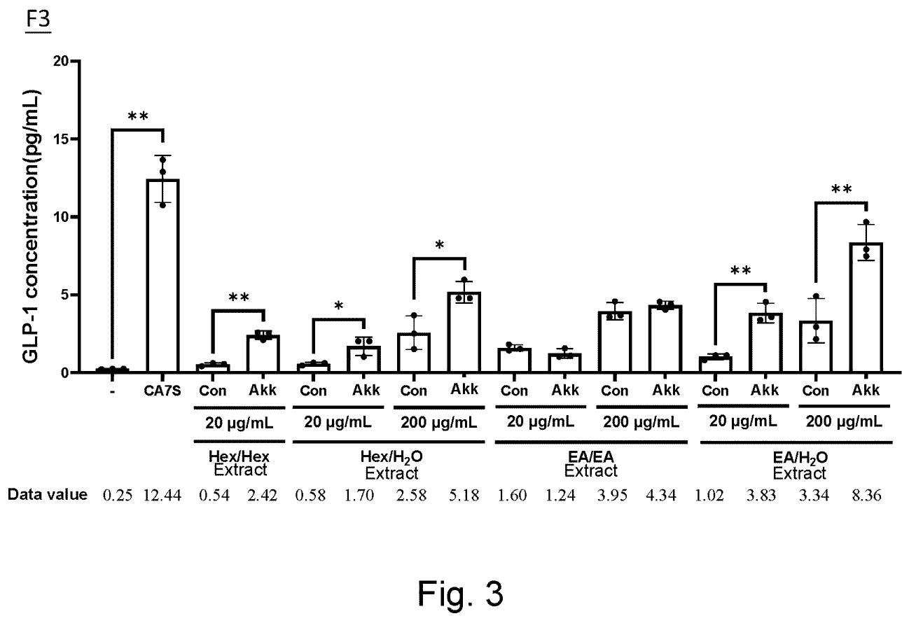

The results are shown in drawing F3 of FIG. 3, after treating different groups of NCI-H716 cells with different extracts for 2 hours, statistical analysis showed that the Hex/Hex, Hex/H₂O and EA/H₂O extracts in AKK BAA-835 culture medium induced GLP-1 secretion in NCI-H716 cells to be significantly more effective than the same extract in uncultured culture medium.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A composition, comprising:
an extract of Akkermansia muciniphila BAA-835 prepared by the following steps comprising:
providing a bacterial culture medium, wherein the bacterial culture medium comprising Akkermansia muciniphila BAA-835 (ATCC BAA-835);
centrifuging the bacterial culture medium to obtain a supernatant and a precipitate;
extracting a portion of the supernatant with ethyl acetate to obtain an ethyl acetate aqueous layer;
drying the ethyl acetate aqueous layer to obtain an ethyl acetate aqueous layer extract;
extracting a remaining portion of the supernatant with n-hexane to obtain a n-hexane aqueous layer and a n-hexane organic layer; and
drying the n-hexane aqueous layer and the n-hexane organic layer respectively to obtain a n-hexane aqueous layer extract and a n-hexane organic layer extract; and
a pharmaceutical or food-acceptable carrier.

2. The composition of claim 1, wherein the composition is a food composition or a pharmaceutical composition.

3. The composition of claim 1, wherein the composition is formulated into capsules, tablets, powders or liquids.

4. A method of preventing and treating overweight or obesity disease comprising administering to a subject in need thereof an effective amount of a composition, wherein the composition comprises an extract of Akkermansia muciniphila BAA-835, wherein the extract of Akkermansia muciniphila BAA-835 prepared by the following steps comprises:
providing a bacterial culture medium, wherein the bacterial culture medium comprises Akkermansia muciniphila BAA-835 (ATCC BAA-835);
centrifuging the bacterial culture medium to obtain a supernatant and a precipitate;
extracting a portion of the supernatant with ethyl acetate to obtain an ethyl acetate aqueous layer;
drying the ethyl acetate aqueous layer to obtain an ethyl acetate aqueous layer extract;
extracting a remaining portion of the supernatant with n-hexane to obtain a n-hexane aqueous layer and a n-hexane organic layer; and
drying the n-hexane aqueous layer and the n-hexane organic layer respectively to obtain a n-hexane aqueous layer extract and a n-hexane organic layer extract.

5. The method of claim 4, wherein the composition is a food composition or a pharmaceutical composition.

6. The method of claim 4, wherein the composition is formulated into capsules, tablets, powders or liquids.

7. The method of claim 4, wherein the composition is administered to a subject orally or parenterally.

8. The method of claim 4, wherein the overweight or the obesity disease is caused by diet.

9. The method of claim 4, further comprising increasing GLP-1 level.

10. The method of claim 4, further comprising reducing triglyceride level.

11. The method of claim 4, wherein the composition further comprises a pharmaceutical or food-acceptable carrier.

12. A method for increasing GLP-1, reducing triglyceride, or a combination thereof, comprising administering to a subject in need thereof an effective amount of a composition, wherein the composition comprising an extract of Akkermansia muciniphila BAA-835, wherein the extract of Akkermansia muciniphila BAA-835 prepared by the following steps comprises:
providing a bacterial culture medium, wherein the bacterial culture medium comprises Akkermansia muciniphila BAA-835 (ATCC BAA-835);
centrifuging the bacterial culture medium to obtain a supernatant and a precipitate;
extracting a portion of the supernatant with ethyl acetate to obtain an ethyl acetate aqueous layer;
drying the ethyl acetate aqueous layer to obtain an ethyl acetate aqueous layer extract;
extracting a remaining portion of the supernatant with n-hexane to obtain a n-hexane aqueous layer and a n-hexane organic layer; and
drying the n-hexane aqueous layer and the n-hexane organic layer respectively to obtain a n-hexane aqueous layer extract and a n-hexane organic layer extract.

13. The method of claim 12, wherein the composition is a food composition or a pharmaceutical composition.

14. The method of claim 12, wherein the composition is formulated into capsules, tablets, powders or liquids.

15. The method of claim 12, wherein the composition is administered to a subject orally or parenterally.
